(19) United States
(12) Patent Application Publication
(10) Pub. No.: US 2007/0055359 A1
(43) Pub. Date: Mar. 8, 2007

(54) Vascular graft marker

(76) Inventors: Stephen C. Messer, Durham, NC (US); Jesse G. Crossen, Durham, NC (US); Robert H. Messier JR., Lynchburg, VA (US); Kevin P. Webb, Durham, NC (US); Jonathan F. Kuniholm, Durham, NC (US); Jason M. Stevens, Calypso, NC (US)

Correspondence Address: HUTCHISON LAW GROUP PLLC, PO BOX 31686, RALEIGH, NC, 27612, US

(21) Appl. No.: 11/509,934

(22) Filed: Aug. 25, 2006

Related U.S. Application Data
(60) Provisional application No. 60/711,466, filed on Aug. 26, 2005.

(51) Int. Cl. A61F 2/06

(52) U.S. Cl. 623/1.34; 623/1.36

(57) ABSTRACT

A vascular graft marker comprising radiopaque material defining a vascular graft origin site and destination of a vascular graft is provided. The graft marker has at least a portion of its body comprising a radiopaque material for radiographically indicating the vascular graft origin site and at least a portion of the radiopaque material defining a textual indicator for radiographically indicating a destination of the vascular graft.

### CROSS-REFERENCE TO RELATED APPLICATION

This application claims the benefit of U.S. Provisional Application No. 60/711,466, filed Aug. 26, 2005, which is incorporated herein by reference in its entirety.

### FIELD

This invention relates to surgical devices and procedures utilizing a radiopaque marker to provide information about the location and destination of a vascular graft.

### BACKGROUND

Cardiovascular disease (CVD) is the most significant medical problem facing Americans today. CVD afflicts over 60 million people in the U.S. claiming more lives (2,600 per day) than the next six leading causes combined. Coronary artery disease in its most severe form leads to blockage of the arteries that supply blood to the heart, which may cause a heart attack or stroke.

Coronary artery bypass graft surgery (CABG) uses a transplanted vessel to bypass the blockage and restore blood flow. It is estimated that approximately two million coronary artery bypass grafts (CABGs) were performed worldwide during more than 800,000 coronary bypass operations. When performing coronary artery bypass surgery, a surgeon may graft a vein taken from another location in the body to the aorta. The graft originates from the aorta and terminates at another location in the heart to reestablish blood supply to those locations where the existing coronary arteries may be obstructed or failing. After the graft is in place, it may be necessary to examine the graft at a future date to assess its patency. This may be done by a cardiologist using medical imaging techniques. For example, a catheter, radiopaque dye and a fluoroscope may be used such that fully invasive surgery may not be required. Fluoroscopy is an imaging technique commonly used to obtain real-time images of the internal structures of a patient through the use of a fluoroscope. Because fluoroscopy uses x-rays, a form of ionizing radiation, it requires that the potential risks from a procedure, such as angioplasty, be carefully balanced with the benefits of the procedure to the patient. While low dose rates of radiation are used during fluoroscopy procedures, the length of a typical procedure may result in a relatively high absorbed dose of radiation by the patient. Although recent advances may reduce the radiation dose to the patient still further, it is desirous to minimize the duration of exposure of the patient and health care professional to high energy radiation.

Subsequent blockage of the grafted vessel at these sites means that further diagnostic testing may be required. Angiography is the primary diagnostic tool used by cardiologists to investigate the patency of coronary arteries and anastomosis graft sites. In post-surgical diagnostic tests using fluoroscopic angiography, a cardiologist inserts a catheter into an artery, for example, in the leg, and routes it to the heart, releasing a radiopaque dye to display blood flow.

Radiopaque graft markers placed at an origin of a graft site where blood enters the graft may allow cardiologists to find the origin of the graft site and insert dye with the catheter and monitor blood flow of the graft. This investigation frequently involves the use of contrast dye that is injected into the graft, time and exposure to x-ray radiation, and probing of the catheter tip against the walls of the aorta. In addition, there may be several vascular grafts on an aorta and determining which graft origin site leads to which of the grafts may not be possible without investigation of each of the grafts. However, a cardiologist without any indication of where the graft destination sites are may be required to probe each site. This probing is risky and may dislodge calcified particles, causing stroke, heart attack, and other circulatory problems.

U.S. Pat. No. 4,693,237 discloses a surgical procedure for radiographically identifying each site and destination of a surgical bypass graft comprising providing a plurality of marker members. The patent discloses that each of the marker members have a different geometrical configuration adapted to identify a different preselected graft destination.

However, it is desirable to provide a vascular marker with a body that comprises radiopaque material that readily identifies the origin of a graft site and at least a portion of the radiopaque material indicating a destination of the graft with textual indicators rather than geometrical shape.

### SUMMARY

In one embodiment, a vascular graft marker is provided. The vascular graft marker comprises a body, the body comprising a radiopaque material for radiographically indicating a vascular graft origin site, at least a portion of the radiopaque material defining a textual indicator for radiographically indicating a destination of the vascular graft.

In another embodiment, a surgical procedure for providing a vascular graft marker radiographically indicating a vascular graft origin site and a destination of a vascular graft in a subject is provided. The procedure comprises providing a graft marker, the graft marker comprising a body having a shape adapted to at least partially adjoin a vascular graft site, at least a portion of the body comprising a radiopaque material for radiographically indicating the vascular graft origin site. The radiopaque material further defines a textual indicator for radiographically indicating a destination of the vascular graft. The procedure comprises positioning the graft marker at the vascular graft origin site such that the graft marker at least partially adjoins the vascular graft origin site and securing the graft marker to the vascular graft origin site to radiographically indicate an origin and a destination of the vascular graft.

In yet another embodiment, a method of determining a vascular graft origin site and destination within a subject is provided. The method comprises providing a subject with a graft marker, the graft marker comprising a body having a shape adapted to at least partially adjoin a vascular graft site, at least a portion of the body comprising a radiopaque material for radiographically indicating the vascular graft origin site, the radiopaque material further defining a textual indicator for radiographically indicating a destination of the vascular graft. The graft marker is positioned such that the graft marker at least partially adjoins the vascular graft origin site in the subject. The method comprises determining the origin and the destination of the vascular graft by radiographically detecting the radiopaque material of the graft marker in the subject.

### BRIEF DESCRIPTION OF THE FIGURES

FIG. 1 is a perspective view showing a vascular graft marker embodiment.

FIG. 2 is a perspective view showing another vascular graft marker embodiment.

FIG. 3 is a perspective view showing another vascular graft marker embodiment.

FIG. 4 is a perspective view showing another vascular graft marker embodiment.

### DETAILED DESCRIPTION

The present invention relates to vascular graft markers, surgical procedures related to graft markers and methods of using graft markers. The vascular graft marker comprises a body comprising a radiopaque material for radiographically indicating a vascular graft origin site and at least a portion of the radiopaque material defining a textual indicator for radiographically indicating a destination of the vascular graft.

The “vascular graft origin site” generally refers to a region where the end of the graft where blood enters (e.g., from the aorta) is attached to adjoining tissue. The vascular graft origin site includes a portion of the graft and/or a portion of the adjoining tissue to which the graft is attached.

The “destination” of the vascular graft generally refers to where the graft terminates at another location.

As used herein, the term “radiography” and its grammatical equivalents, for example, radiographically and radiographic, refers to medical imaging techniques by which an area of a subject's body that is not externally visible is observed using ionizing radiation, non-ionizing radiation or other imaging techniques. The medical imaging technique may create analog or digital images for medical diagnoses. By way of example, radiography includes x-rays, fluoroscopy, magnetic resonance imaging (MRI) and computed axial tomography (CAT).

The shape of the vascular graft marker body may be of any shape such as rectangular, substantially circular or completely circular. The body of the graft marker may be made from any suitable material such as metal, non-metal, inert plastic or like resinous material. Such plastic inert materials are known in the medical arts and are generally known and provided as approved plastics for use within the human body by the Federal Food and Drug Administration. For example, the body may include a biocompatible material. The biocompatible material may be comprised of polysilicone, polyurethane, or polyolefin.

The body of the graft marker may be rigid or flexible, or may be of any degree of flexibility between these extremes as desired. Polymer processing aids, fillers, additives, oils and the like may be added to the material used to form the body. The body of the graft marker may be formed, for example, by liquid injection molding (LIM) or other injection, compression or extrusion methods, or may be cut from bulk sheet stock. The body of the graft marker may be comprised completely of radiopaque material, encapsulated radiopaque material or combinations thereof. The radiopaque material may be uniformly distributed throughout the graft marker body. Radiopaque material may be encapsulated inside the biocompatible material using any suitable process, for example, in-mold decoration (IMD). The radiopaque material may be externally applied to the body of the marker using, for example, a pad printing processes. The IMD process may advantageously provide for complete encapsulation of the radiopaque material in the marker.

At least a portion of the body comprises a radiopaque material for radiographically indicating the vascular graft origin site. The distribution of radiopaque material indicating the vascular graft origin site may be provided in any pattern. For example, the radiopaque material contained within the graft marker may be arranged in any shape such that when radiographically examined the radiopaque material indicates the origin of the graft. The marker may adjoin the graft origin site or may at least partially encircle the graft origin site and clearly indicate through the radiologically visible radiopaque material the location of the graft origin. By way of example, radiographic material arranged in a circular shape or other shape in the body of the graft marker may radiographically present as a circular region or other shape about the vascular graft site thereby indicating the graft origin. As another example, the radiopaque material defining the textual indicators may be used to indicate the origin of the graft site.

At least a portion of the radiopaque material defines a textual indicator for radiographically indicating a destination of the vascular graft. The presence of radiopaque material in the body of the graft marker may be arranged in the shape of text or other symbols such that the graft marker may present the textual indicators as a positive radiographic image to indicate the destination of the graft. The radiopaque material may also be positioned such that an absence of radiopaque material in the body of the graft marker may present the textual indicators as a negative radiographic image to indicate the destination of the graft. The textual indicators may include any alpha-numeric character, punctuation, hyphenation or textual symbols and may include combinations thereof. The textual indicator may readily identify to a surgeon, doctor or cardiologist the origin and/or the destination of the graft without reference to a coded meaning of the textual indicator.

Preferred textual indicators are alpha-numeric characters with or without hyphenation. More preferred textual indicators may be acronyms or words readily identifiable to a surgeon, cardiologist or doctor. By way of example, an alpha-numeric character may be an acronym representing a portion of a cardiovascular system, which may be a destination of a graft, for example, a bypassed artery. Thus, if the artery bypassed is the right coronary artery, then the textual indicator may provide the letters “RCA”. Any acronyms commonly used by surgeons or cardiologists may be used in the vascular graft marker herein disclosed to designate the destination of the graft. Non-limiting examples of acronyms commonly used in the field of cardiology are presented in Table 1.

| Textual Acronym | Common Meaning |
|---|---|
| LAD | Left anterior descending artery. Branch off the |
| | left main coronary artery that provides |
| | oxygenated blood to both ventricles. |
| LAD-D1 | Left anterior descending artery 1^{st} diagonal. |
| LAD-D2 | Left anterior descending artery 2^{nd} diagonal. |
| LAD-RAMUS | Left anterior descending artery branch. |
| RAMS | Branch. |
| OMB | Obtuse marginal branch. Branch of the |
| | circumflex artery that supplies the left ventricle |
| | with oxygenated blood. |
| OM1 | Obtuse marginal 1 |
| OM2 | Obtuse marginal 2 |
| OM3 | Obtuse marginal 3 |
| OM1-OM2 | Obtuse marginal 1 and obtuse marginal 2 |
| OM2-OM3 | Obtuse marginal 2 and obtuse marginal 3 |
| OM1-OM3 | Obtuse marginal 1 and obtuse marginal 3 |
| RCA | Right coronary artery. Main coronary vessel |
| | that branches off the aorta and supplies the right |
| | atrium of the heart with oxygenated blood; |
| | divides into the posterior descending artery. |
| PDA | Posterior descending artery. Branch of the right |
| | coronary artery that supplies both ventricles of |
| | the heart with oxygenated blood. |
| PL | Posterolateral. |
| RVB | Right ventricular branch. |
| RCA-PDA | Right coronary artery and posterior descending |
| | artery |
| RCA-PL | Right coronary artery and posterolateral |
| RVB-PL | Right ventricular branch and posterolateral |

Any radiopaque material may be used, including, for example, barium, bismuth, cesium, silver, thorium, tin, tantalum, tungsten, zirconium, iodine compounds, and lanthanide compounds. Barium sulfate is generally preferred as radiopaque material because of its low biological solubility and low absorbability.

The graft marker may be adapted to be secured to a vascular graft origin site. Any suitable means to secure the graft marker to the vascular graft origin site may be used, for example, sutures, adhesives, staples, magnets, and the like. The graft marker may be secured, for example, in the case of a coronary bypass, to the aorta at the graft origin site. The same sutures that are used to secure the graft to the aorta may be used to secure the marker.

A surgical procedure for providing a vascular graft marker radiographically indicating an origin and destination is provided. The procedure comprises providing a graft marker as herein disclosed and positioning the graft marker such that it at least partially adjoins a vascular graft origin site. The graft marker is secured to the vascular graft origin site. Any suitable means to secure the graft marker to the vascular graft origin site may be used such as sutures, adhesives, staples, magnets, and the like. The secured graft marker provided by this procedure radiographically indicates an origin and a destination of the vascular graft. Preferably, the procedure is directed to a coronary vascular graft, for example, a coronary artery bypass.

A method of determining a vasculature graft origin and destination within a subject is also provided. The method is useful for determining the origin and destination of a vascular graft in a subject having a graft marker as herein disclosed. Thus, a graft marker of the subject, being positioned such that the graft marker at least partially adjoins a vascular graft origin site in the subject to indicate the origin of a vascular graft, may be radiographically examined to detect the radiopaque material and to determine the destination of the vascular graft. By way of example, the textual indicator defined by the radiopaque material may be radiographically detected using x-rays, fluoroscopy, magnetic resonance imaging (MRI) or computed axial tomography (CAT). The method is particularly preferred for when the vascular graft is a coronary artery bypass graft.

The graft marker herein disclosed may be particularly suited for use in the treatment of coronary thrombosis of a coronary artery. Such treatment may be necessary to treat or possibly reduce myocardial infarction and may be performed on patients with coronary artery disease, atherosclerosis or angina.

Angiography, for example, fluoroscopic angiography, may be used in combination with the graft marker described herein to find grafts, to guide placement of catheters, and determine the destination of the graft. Fluoroscopic angiography maybe used in combination with the graft marker described herein to diagnose leg, heart and cerebral vessels and grafts thereof. Particularly preferred is the use of the graft marker herein disclosed with fluoroscopy for coronary bypass grafts. In addition to the applications above, the graft marker described herein may be useful in cerebral, orthopedical and urological vasculature.

It may be desirable to place a graft that supplies more than one location on the heart with blood and thus has multiple destinations yet only one graft origin site. Use of more than one vascular marker to mark multiple graft destinations from one graft origin site is therefore envisaged. Thus, two or more radiopaque markers as described herein may be layered together with their designation areas separated radially or spatially in order to make each designation area distinct from the other designations in the layers of graft markers. Alternatively, a marker may be provided with multiple destinations provided in the same marker.

The probing of an aorta for graft sites and graft destinations is risky and may dislodge calcified particles, causing stroke, heart attack, and other circulatory problems. In addition to these aforementioned risks, considerable time is expended searching for the graft site, resulting in increased exposure to electromagnetic radiation and larger doses of the radiopaque dye. Each of these effects is undesirable, and may be reduced or eliminated with markers as described herein that provide information about the origin of the graft and readily identifiable information about the destination of the graft.

Thus, the graft markers herein disclosed placed at a graft origin site may allow quick routing of the catheter to the site and may significantly reduce the risks of catheterization and radiation exposure to patients and health care professionals. By providing destination information as well as origination information hazards in follow-up catherization may be reduced or eliminated. This may allow a reduction in time, cost, contrast agent, and radiation exposure to the doctor, staff and patient. The doctor may be able to identify the destination graft readily (and without the use of a coding system) and may be able to avoid or eliminate investigating grafts of no interest. This ability to locate a specific graft of interest may also result in less physical movement and probing with the catheter tip, which may decrease the risk that material may be dislodged and migrate to another part of the body and cause harm. For example, if a patient surgically implanted with the vascular graft marker herein described (e.g., indicating RCA as the destination of the graft in the patient) is diagnosed with an irregularity in the right atrium, the doctor would first check the patency of the right coronary artery (RCA) bypass graft which supplies blood to this region. This may preclude the necessity to investigate other vessels at all. Without a graft marker indicating origin and destination, the doctor may be required to check each of the graft sites until the appropriate vessel to be investigated is found.

The graft markers herein disclosed that do not completely encircle the vessel may allow installation off of heart-lung machines, greatly reducing risk to the patient. Other applications may benefit from or be realized by direct readable information embedded in the body. Markers with similar embedded and/or directly readable text may be constructed in a variety of shapes as needed to allow use in various procedures.

The markers herein disclosed may be individually packaged to allow the surgery team to select the marker needed and prepare it for the procedure. The device may be sterilized in the package or prior to use at the surgical site as desired. Alternatively, the marker may be packaged in groups of all possible markers and combinations of markers. The particular required markers could be removed from the packaging for use as needed without the need for preplanning.

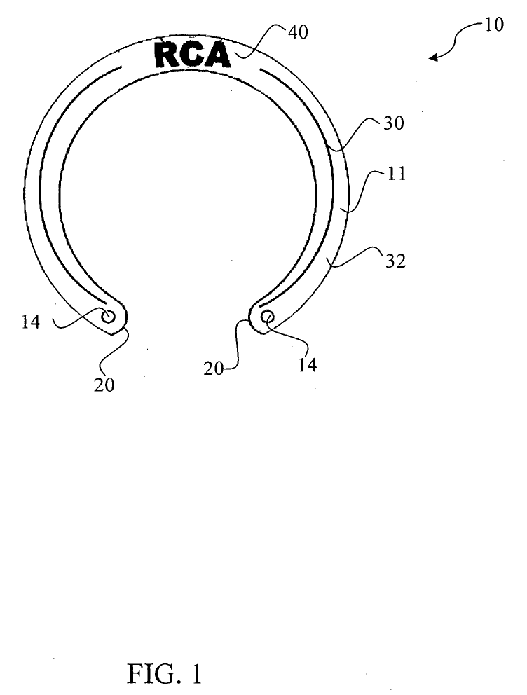

Referring now to the drawings, various illustrative embodiments will be described. The graft marker 10 as shown in FIG. 1 provides for example, a horseshoe shape. Marker 10 is comprised of a marker body 11 which comprises a concentric distribution of radiopaque material 30. Surrounding and embedding this concentric distribution of radiopaque material 30 is an outer material 32. Outer material may be formed from a biocompatible material comprising a radiopaque material. The biocompatible material may further comprise polysilicone, polyurethane, or polyolefin.

As depicted in FIG. 1, a pair of openings 14 at the free ends 20 of the ring provide for the attachment of the marker to a vascular graft origin site, for example, with sutures. Openings 14 may be positioned anywhere on the marker. Such a surgical attachment of marker 10 to a vessel wall or vascular graft origin site may be performed by a surgeon simultaneously with the removal of the graft vein, for example, from the leg or vessel during an operation such as a coronary bypass operation. Alternatively, marker 10 may be secured after the graft is sutured into its grafted position. The latter providing for the graft marker 10 to be advantageously installed after removing a patient from a heart-lung machine.

Sutures may be placed in any location on the marker at the discretion of the surgeon. The size of openings 14 is such as to easily pass a suture material as well as a needle such that they are fastened in fixed position to the tissue at the site of an anastomosis to indicate the origin of the graft.

At least a portion of the radiopaque material defines at least one textual indicators 40. The radiopaque material forming the indication of the graft origin site and the textual indicator representing the destination may present as a positive radiographic image. Thus, a ring shape and textual indicators 40 of graft marker 10 may appear as bright images on a dark background.

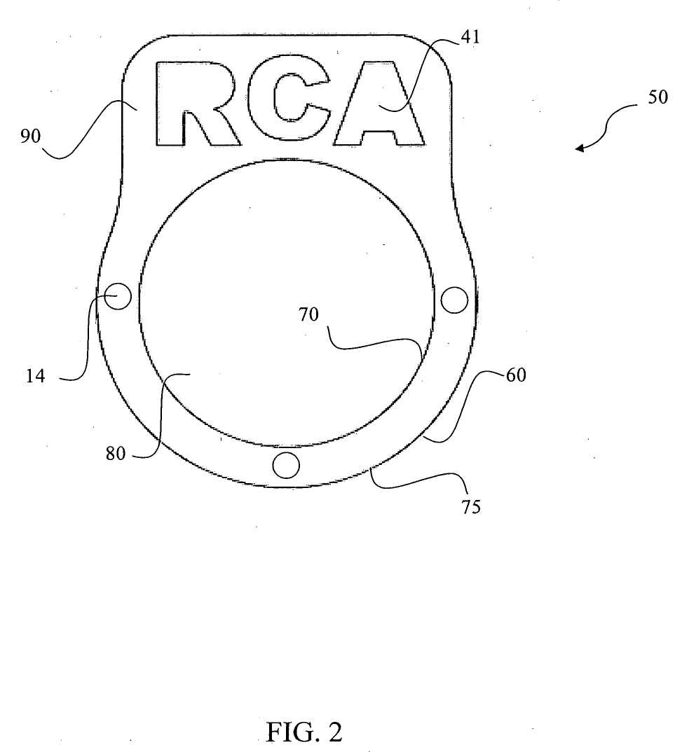

Referring now to FIG. 2, graft marker 50 will be seen to take the general form of a closed ring, for example, a washer-shape, adapted to at least partially encircle a vascular graft origin site. The washer-shaped graft marker 50 has a body 75 with an outer diameter 60 and a smaller inner diameter 70, the inner diameter further defining a void 80. The body 75 of the graft marker 50 may be metal or non-metal. The body 75 of graft marker 50 may be formed from material such as a biocompatible material comprising a radiopaque material.

The graft marker 50 comprises radiopaque material that may be uniformly distributed throughout at least a portion of the washer-shape to define an origin of the vascular graft site such as at least a portion of a ring-shape. The washer-shaped graft marker comprises a flange-like area 90 projecting outwardly from the outer diameter 60 of the washer-shaped graft marker 50. More than one flange-like area may be provided on the graft marker.

The radiopaque material in the flange-like area 90 defines at least one textual indicator 41 or at least one textual indicator may be formed from the absence of material in the flange-like area. Thus, graft marker 50 may present a positive image of at least a portion of a ring shape and a positive image for the textual indicators 41 when the radiopaque material defines a textual indicator. Alternatively, graft marker 50 may present a positive image of at least a portion of a ring shape and a negative image for the textual indicators 41 when the absence of radiopaque material defines a textual indicator.

The textual indicators 41 and/or the entire shape of the body 75 may be formed, for example, by die-stamping a shape from a uniform sheet stock. Alternatively, the textual indicator and body shape forming may be accomplished by laser cutting or some other means of controlled material removal.

Graft marker 50 is adapted to be secured to a vascular graft origin site. Any suitable means to secure the graft marker to the vascular graft origin site may be used such as sutures, adhesives, staples, magnets, and the like. As depicted in FIG. 2, openings 14 are provided on the washer-shape graft marker 50. The openings 14 may be positioned anywhere on the marker. The size of openings 14 is such as to easily pass suture material as well as a needle such as to secure the graft in fixed position to tissue at a site, for example, a vascular graft origin site to indicate the origin of the graft.

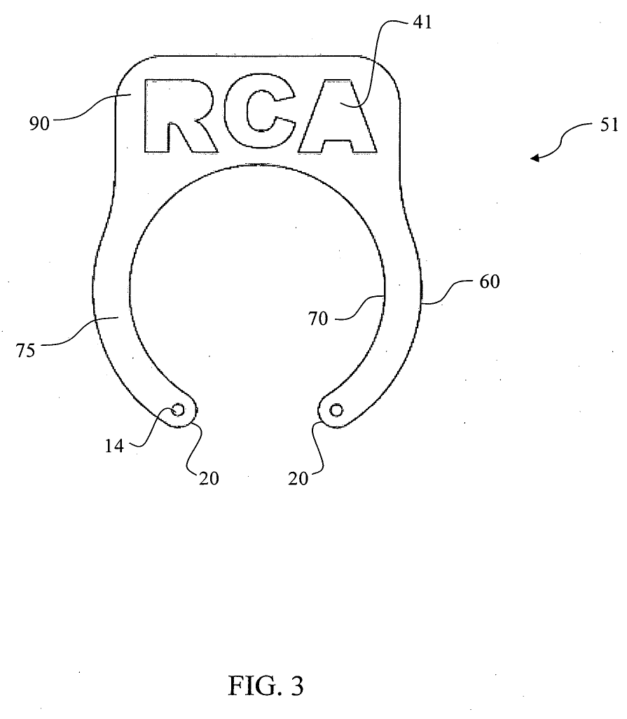

Graft marker 50 forms a continuous ring and may be placed on the graft before the graft is attached. If this is neglected, or if for any reason the surgeon desires to place the marker at the vascular graft origin site after completion of a vascular graft procedure, graft marker 50 may be cut using a variety of tools available in the surgical arena. This will allow the marker to be opened and placed around a graft that is already in place. Alternatively, graft marker device 50 may further include a radial slit (not shown). As shown in FIG. 3, which depicts vascular graft marker 51, an opening positioned between ends 20 of body 75, may facilitate at least partially encircling the vascular graft, for example, after the origin and/or destination of the graft are secured. The vascular graft marker 50 or 51 may be spread apart along a slit or opening of the marker and the vascular graft may be at least partially encircled. The graft marker 50 or 51 may advantageously allow for installation of a graft marker after removing a patient from a heart-lung machine.

The graft marker 50 or 51 may be manipulated into a desired position in contact with or adjoining the vascular graft origin site. By way of example, the graft marker 50 or 51 may be positioned around a bypass graft artery and in contact with a vascular graft origin site on the aorta. The graft marker may be secured to the graft origin site which may include an area of the aorta or heart muscle.

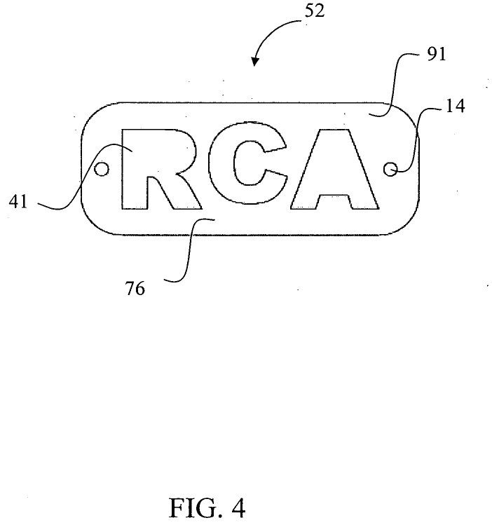

The graft marker 50 or 51 thus provides for a radiographic guide which may be used, for example, to accurately locate the coronary graft during an angiographic procedure. Preferably the radiopaque material which may be uniformly distributed throughout the body 75 of the graft marker 50 or 51 provides any shape that when radiographically examined may indicate the vascular graft origin site. Alternatively, as shown in FIG. 4, vascular graft marker 52, the body 76 of which is shaped to accommodate essentially textual indicators and openings 14 in rectangular area 91 may indicate the vascular graft origin site and the destination when radiographically examined. Thus, the origin site and destination site may both be indicated with a graft marker body comprising radiopaque material defining textual indicators.

When the radiopaque material of graft marker 50, 51 or 52 is uniformly distributed throughout the graft marker, the absence of material arranged in the shape of text or other symbols, for example, alpha-numeric characters, in the flange-like area 90 or rectangular area 91 may radiographically present at least one textual indicators 41 as a negative image when radiographically examined. The graft marker 50, 51 or 52 may present under fluoroscopy, for example, as a positive image indicating the origin of the graft site with a contrasting negative image of the destination, for example, textual indicators 41.

Alternatively, the textual indicator may be formed from the presence of radiopaque material in the flange-like area 90 or rectangular area 91 and present at least one textual indicators 41 as a positive image indicating the destination of the graft when radiographically examined. The graft marker 50, 51 or 52 may present under fluoroscopy, for example, as a positive image indicating the origin of the graft with a positive image of the destination, for example textual indicators 41.

While the invention has been described in detail and with reference to specific embodiments thereof, it will be apparent to one skilled in the art that various changes and modifications can be made without departing from the spirit and scope of the invention.

We claim:

1. A vascular graft marker comprising:
a body, at least a portion of the body comprising a radiopaque material for radiographically indicating a vascular graft origin site, at least a portion of the radiopaque material defining a textual indicator for radiographically indicating a destination of the vascular graft.

2. The graft marker of claim 1, wherein the body of the graft marker least partially encircles the vascular graft origin site.

3. The graft marker of claim 1, wherein the body of the graft marker completely encircles the vascular graft origin site.

4. The graft marker of claim 1, wherein the graft marker is rectangular.

5. The graft marker of claim 1, wherein the graft marker is horseshoe-shaped.

6. The graft marker of claim 1, wherein the graft marker is washer-shaped.

7. The graft marker of claim 1, wherein the graft marker is adapted for securing to the vascular graft origin site.

8. The graft marker of claim 1, wherein the graft marker is adapted to be sutured to the vascular graft origin site.

9. The graft marker of claim 1, wherein the radiopaque material defining the origin and destination of the vascular graft origin site is detectable by fluoroscopy.

10. The graft marker of claim 1, wherein the radiopaque material radiographically indicates the textual indicator positively.

11. The graft marker of claim 1, wherein the radiopaque material radiographically indicates the textual indicator negatively.

12. The graft marker of claim 1, wherein the radiopaque material is substantially uniformly dispersed throughout the graft marker.

13. The graft marker of claim 6, wherein the washer-shaped graft marker has an outer diameter and a smaller inner diameter, the inner diameter further defining a void, the washer-shaped graft marker further comprising at least one flange-like area projecting outwardly from the outer diameter of the washer-shaped graft marker.

14. The graft marker of claim 13, wherein the at least one flange-like area comprises the radiopaque material defining the textual indicator.

15. The graft marker of claim 1, wherein the textual indicator is an acronym.

16. The graft marker of claim 15, wherein the acronym represents a portion of a cardiovascular system.

17. The graft marker of claim 1, wherein the body of the graft marker is metal or non-metal.

18. The graft marker of claim 1, wherein the body of the graft marker is a biocompatible material.

19. The graft marker of claim 1, wherein the body of the graft marker further comprises polysilicone, polyurethane, or polyolefin.

20. The graft marker of claim 1, wherein the radiopaque material is barium sulfate.

21. A surgical procedure for providing a vascular graft marker radiographically indicating a vascular graft origin site and a destination of a vascular graft in a subject, the procedure comprising:
providing a graft marker, the graft marker comprising a body having shape adapted to at least partially adjoin a vascular graft origin site, at least a portion of the body comprising a radiopaque material for radiographically indicating the vascular graft origin site, the radiopaque material further defining a textual indicator for radiographically indicating a destination of the vascular graft;
positioning the graft marker at the vascular graft origin site such that the graft marker at least partially adjoins the vascular graft origin site; and
securing the graft marker to the vascular graft origin site to radiographically indicate an origin and a destination of the vascular graft.

22. The procedure of claim 21, wherein the vascular graft is a coronary artery bypass graft.

23. A method of determining a vascular graft origin site and destination within a subject, the method comprising:
providing a subject with a graft marker, wherein the graft marker comprises a body having shape adapted to at least partially adjoin a vascular graft origin site, at least a portion of the body comprising a radiopaque material for radiographically indicating the vascular graft origin site, the radiopaque material further defining a textual indicator for radiographically indicating a destination of the vascular graft, wherein the graft marker is positioned such that the graft marker at least partially adjoins a vascular graft origin site in the subject; and
determining the origin site and destination of the vascular graft by radiographically detecting the radiopaque material of the graft marker in the subject.

24. The method of claim 23, wherein the vascular graft is a coronary artery bypass graft.
